(19) United States
(12) Patent Application Publication
(10) Pub. No.: US 2023/0248718 A1
(43) Pub. Date: Aug. 10, 2023

(54) PHARMACEUTICAL COMBINATION OF ANTISPASMODIC AND ANXIOLYTIC AGENT

(71) Applicant: Sushma Paul BERLIA, New Delhi (IN)

(72) Inventors: Sushma Paul BERLIA, New Delhi (IN); Nishant BERLIA, New Delhi (IN); Aditya BERLIA, New Delhi (IN); Gurvinder SINGH, New Delhi (IN)

(21) Appl. No.: 18/107,619

(22) Filed: Feb. 9, 2023

(30) Foreign Application Priority Data
Feb. 9, 2022 (IN) 20221 1006919

(51) Int. Cl. A61K 31/472; A61K 31/5513; A61K 9/20

(52) U.S. Cl. CPC A61K 31/472; A61K 31/5513; A61K 9/2086

(57) ABSTRACT

The present disclosure provides fixed dose combinations of Drotaverine or a salt thereof and benzodiazepines, methods of preparing fixed dose combinations, and their use in treatment.

### FIELD

The present disclosure relates to a pharmaceutical combination of an antispasmodic and an anxiolytic agent. The disclosure provides stable formulations comprising Drotaverine and Chlordiazepoxide or salts thereof. The disclosure also relates to methods for preparation of such combination and formulations and to uses thereof.

Irritable bowel syndrome (IBS) is a common functional bowel disorder mainly characterized by recurrent abdominal pain associated with defecation and changes in stool form or frequency. IBS is classified into four subtypes according to abnormal bowel habits: diarrhoea predominant IBS (IBS-D), constipation-predominant IBS (IBSC), mixed-type IBS (IBS-M), or unclassified IBS. About 10% of the global population suffers from some type of IBS.

The exact cause of IBS is still unclear, but several mechanisms have been implicated. Symptoms may result from a disturbance in the way the gut, brain, and nervous system interact. This can cause changes in normal bowel movement and sensation. At least half of the IBS patients are described as depressed, anxious, or hypochondriac as IBS is a disorder of bowel motility and sensation that is exacerbated by psychosocial stressors. Abdominal pain is nearly ubiquitous in patients with IBS, significantly affecting the patient's quality of life. A report by the Indian Society of Gastroenterology task force revealed that as high as 70% of IBS patients have abdominal pain or discomfort.

The cause of abdominal pain has traditionally been ascribed to smooth muscle spasm. Therefore, antispasmodics have been and remain the mainstay of therapy. Antispasmodic agents relax the smooth muscle of the gut or reduce its contractility. Patients with IBS typically have augmented colonic motility in response to meals (gastrocolic reflex), which can be associated with diarrhoea and cramps.

When bowel-directed therapies for diarrhoea or constipation are inadequate or when pain is a prominent finding, therapy with antispasmodic drugs is typically useful, particularly for postprandial symptoms. Antispasmodic agents can reduce this excessive postprandial contractility. Data from randomized trials indicate that antispasmodic agents decrease global symptoms and reduce pain. Since IBS is a disorder of bowel motility and sensation that is exacerbated by psychosocial stressors, treatment is most successful when a multicomponent, comprehensive approach is used. Indeed, therapies for IBS are generally directed at gastrointestinal motor, gastrointestinal sensory, or central nervous system processing.

Drotaverine is an antispasmodic agent with excellent tolerability and is available in more than 42 countries worldwide. Drotaverine has spasmolytic and vasodilating action. It produces antispasmodic action by inhibiting phosphodiesterase activity. Drotaverine has a fast onset of action that initiates relief from pain within 12 minutes. Drotaverine is an effective antispasmodic and does not produce anticholinergic side effects. Drotaverine has been used to relieve pain in IBS. Drotaverine brings relief of spasm in IBS by its unique site-specific action and causes normalisation of smooth muscle contraction. Thus, it does not cause constipation, making it suitable for use in all the types of IBS (Diarrhoea predominant IBS, Constipation predominant IBS, unclassified IBS, and mixed IBS). Drotaverine is available as tablets of 40 mg and 80 mg strength. The usual adult dose of Drotaverine is 120-240 mg daily (in 2-3 divided doses).

In a pharmacokinetic and bioavailability study, Drotaverine has shown rapid absorption, with an onset of action within 12 minutes after oral administration of 80 mg strength IR tablets and almost 90% of drug absorption from small intestine. The peak plasma levels were obtained after 1 to 3 hours while elimination half-life ranges from 7 to 11.95 hours. Toxicological studies report oral LD50 for Drotaverine as >1000mg/Kg body weight, and Drotaverine has wide therapeutic index.

Anxiolytic agents are medications that can affect the entire body, including effects on mood and the gastrointestinal system. Benzodiazepines are a class of anxiolytic agents.

Chlordiazepoxide hydrochloride is a benzodiazepine that has antianxiety, sedative, appetite-stimulating, and weak analgesic actions. It works by increasing the action of a chemical messenger (GABA) which suppresses the abnormal and excessive activity of the nerve cells in the brain. Mechanism of action includes binding of the drug to stereo-specific benzodiazepine binding sites on GABA receptors at several sites within the central nervous system, which results in an increased binding of the inhibitory neurotransmitter GABA to the GABA (A) receptor. Hence benzodiazepines enhance GABA mediated chloride influx which results in membrane hyperpolarisation. The neuroinhibitory effect results in sedative, hypnotic, anxiolytic, and muscle relaxant properties. Chlordiazepoxide is among the safer and effective psychopharmacologic compounds available, as demonstrated by extensive clinical evidence. Chlordiazepoxide is available as capsules containing 5 mg, 10 mg, or 25 mg of Chlordiazepoxide HCl.

Chlordiazepoxide HCl takes several hours for peak blood levels to be reached and the half-life of the drug is between 24 and 48 hours. After administration of the drug is discontinued, plasma levels decline slowly over a period of several days. It is excreted in the urine as 1% to 2% being unchanged form and 3% to 6% as conjugate form.

The current recommended dosage schedule of Drotaverine and Chlordiazepoxide is 3 tablets each per day, amounting to 6 tablets per day, if a patient is prescribed both the medications. The number of tablets per day often will directly impact patient compliance. By combining both drugs into a single pill, it is possible to reduce pill burden of the drugs thereby increasing patient compliance. The use of combination formulations is an emerging method to reduce pill burden, particularly in patients with multiple chronic conditions. By reducing overall pill burden and simplifying medication regimens, fixed combinations have shown to improve medication adherence in several studies. However, combining different active ingredients that retain efficacy with no detriment to stability are difficult to formulate.

Fixed dose combination (FDC) is defined as a combination of two or more active pharmaceutical ingredients or compounds formulated as a single medicine irrespective of its dosage form. Each drug in FDC has an independent mode of action and provides synergistic, additive, or complementary pharmacological effects. Patients with IBS frequently experience wide range of comorbidities that contribute to disease burden and are likely prescribed multiple medications. However, keeping track of several dosing schedules for multiple medicines can be a burden for these patients. This can lead to reduced patient compliance with an approved dosing schedule, which in turn leads to decreased efficacy for the medication.

An FDC including a combination of an antispasmodic agent and an anxiolytic agent may be helpful for patients with abdominal pain associated with anxiety or refractory disease. Preparations combining other agents have therefore been introduced and have been generally accepted. One combination used in the treatment of IBS consists of clidinium bromide/mebeverine (antispasmodic agent) and Chlordiazepoxide (anxiolytic agent). Moreover, use of Drotaverine/Mebeverine HCL or Chlordiazepoxide individually in treatment of functional gastrointestinal disorders is known. However, an FDC of Drotaverine and a benzodiazepine is not known in the art.

Some possible combinations of antispasmodic agents with other agents studied in the literature are as follows:

Indian Patent Application 5678/CHE/2014 discloses a fixed dose pharmaceutical formulation of an analgesic and an anti-spasmodic drug (Drotaverine). WO2019149917 relates to a pharmaceutical composition comprising metamizole, Drotaverine, and caffeine. Indian Patent Application 2208/MUM/2013 discloses an FDC of mebeverine and one or more antiflatulents. WO2016041036 relates to a pharmaceutical composition containing an analgesic agent (ketorolac tromethamine) and an antispasmodic agent being hyoscine, pargeverine, tolterodine, mebeverine, or papaverine.

The present disclosure includes the FDC of Drotaverine, as the antispasmodic agent, with the anxiolytic drug Chlordiazepoxide (CDO), which is an attractive solution to provide effective relief of spastic colon and associated symptoms of anxiety and tension in the gastrointestinal tract. By virtue of different mechanisms of action, combination of the two agents can provide comprehensive and rapid relief from pain, spasm, and/or anxiety in patients with IBS.

Accordingly, the inventors of present disclosure conducted experimental studies for combining Drotaverine as antispasmodic agent and Chlordiazepoxide as anxiolytic agent. However, a significant degradation of Chlordiazepoxide and Drotaverine was observed when they were mixed together. Details of experiments conducted and results obtained are described herein below.

There exists a need in the art for a drug delivery format which allows these two classes of drugs to be formulated as an FDC in a single pill for the ease of administration and better patient compliance. A chemically stable pharmaceutical combination or formulation(s) containing Drotaverine and Chlordiazepoxide active drugs is needed.

Also there exists in the art a need for methods to provide suitable pharmaceutical packaging for FDC formulations comprising Chlordiazepoxide or a pharmaceutically acceptable salt thereof and Drotaverine or a pharmaceutically acceptable salt thereof. Use of packaging formats can also impact the stability of these pharmaceutical dosage forms, allowing for longer storage periods.

### SUMMARY OF THE DISCLOSURE

In some embodiments, the present disclosure provides a FDC comprising Drotaverine or a pharmaceutically acceptable salt thereof and a benzodiazepine. In some embodiments, the present disclosure provides an FDC comprising Drotaverine or a pharmaceutically acceptable salt thereof and Chlordiazepoxide or a pharmaceutically acceptable salt thereof. In some embodiments, the FDC comprises a core and a coating surrounding the core, and the core comprises at least one layer. In some embodiments, the core comprises Drotaverine or a pharmaceutically acceptable salt thereof, and the coating surrounding the core comprises Chlordiazepoxide or a pharmaceutically acceptable salt thereof. In some embodiments the core comprises at least two layers, one layer comprises Drotaverine or a pharmaceutically acceptable salt thereof, and a second layer comprises Chlordiazepoxide or a pharmaceutically acceptable salt thereof. In some embodiments, the core further comprises a third layer, wherein the third layer is between the one layer comprising Drotaverine or a pharmaceutically acceptable salt thereof and the second layer comprising Chlordiazepoxide or a pharmaceutically acceptable salt thereof, and the third layer comprises an inert excipient.

In some embodiments, the present disclosure provides an FDC comprising 20-300 mg Drotaverine or a pharmaceutically acceptable salt thereof. In some embodiments, the pharmaceutically acceptable salt of Drotaverine is Drotaverine hydrochloride. In some embodiments, the FDC comprises 2.5-40 mg Chlordiazepoxide or a pharmaceutically acceptable salt thereof. In some embodiments, the pharmaceutically acceptable salt of Chlordiazepoxide is Chlordiazepoxide hydrochloride.

In some embodiments, the present disclosure provides an FDC comprising a coating agent, wherein the coating agent comprises polyvinylpyrrolidone, methyl cellulose, ethyl cellulose, hydroxypropyl cellulose, hydroxypropylmethyl cellulose, sodium carboxymethylcellulose, dextrin, maltodextrin, lactose, D-mannitol, polyvinyl alcohol polymer, methacrylic acid copolymer, aminoalkylmethacrylate copolymer, or ethyl acrylate methyl methacrylate copolymer, or a combination thereof. In some embodiments, the coating agent further comprises a plasticizer, an anti-tacking agent, an opacifier, or a coloring agent, or a combination thereof.

In some embodiments, the present disclosure provides an FDC comprising an acidifying agent, an antioxidant, a diluent, a binder, a surfactant, a lubricant, or a glidant, or a combination thereof. In some embodiments, the acidifying agent comprises citric acid, fumaric acid, lactic acid, maleic acid, malic acid or tartaric acid, or a combination thereof. In some embodiments, the antioxidant comprises butylated hydroxy-anisole (BHA), butylated hydroxytoluene (BHT), sodium metabisulfite, sodium thiosulfate, propyl gallate, ascorbic acid, or cysteine, or a combination thereof. In some embodiments, the diluent comprises lactose monohydrate, lactose anhydrous, mannitol, maize starch, corn starch, pregelatinized starch, starch 1500, crystalline cellulose, powdered cellulose, directly compressible grade cellulose, sorbitol, xylitol, calcium carbonate, magnesium carbonate, dibasic calcium phosphate, or tribasic calcium phosphate, or a combination thereof. In some embodiments, the binder comprises hydroxypropyl cellulose, hydroxypropyl methylcellulose, polyvinylpyrrolidone, copovidone, powdered acacia, gelatin, guar gum, a carbomer, methylcellulose, a polymethacrylate, or a starch, or a combination thereof. In some embodiments, the surfactant comprises a polyoxyethylene-polyoxypropylene block copolymer, an alkyl sulfate, an alkyl aryl sulfonate, polyethylene glycol, or a polysorbate, or a combination thereof. In some embodiments, the lubricant comprises magnesium stearate, glyceryl monostearate, palmitic acid, talc, carnauba wax, calcium stearate, sodium stearate, sodium lauryl sulfate, magnesium lauryl sulfate, zinc stearate, polyoxymethylene monostearate, calcium silicate, silicon dioxide, a hydrogenated vegetable oil or fat, stearic acid, or glyceryl behenate, or a combination thereof. In some embodiments, the glidant comprises colloidal silicon dioxide or talc, or a combination thereof.

In some embodiments, the present disclosure provides an FDC prepared using a direct compression process. In some embodiments, the FDC is prepared using a dry granulation process. In some embodiments, the FDC is prepared using a wet granulation process. In some embodiments, the FDC is prepared using a fluidized bed process. In some embodiments, the FDC is packaged along with a pharmaceutically acceptable desiccant. In some embodiments, the FDC is packaged in an inert gas environment. In some embodiments, the FDC exhibits acceptable chemical stability at six months according to ICH guidelines.

In some embodiments, the present disclosure provides an FDC comprising Drotaverine hydrochloride, Chlordiazepoxide, and Citric acid, wherein the formulation comprises a bilayer core with a coating, wherein Drotaverine hydrochloride is in one layer of the core, and wherein Chlordiazepoxide is in a second layer of the core. In some embodiments, the FDC is prepared using a dry granulation process.

In some embodiments, the present disclosure provides a method of preparing an FDC of Drotaverine or a pharmaceutically acceptable salt thereof and Chlordiazepoxide or a pharmaceutically acceptable salt thereof comprising a core with a coating using a direct compression process, the method comprising: Mixing intragranular ingredients for the core of the FDC; loading the intragranular ingredient on a tableting machine; compressing the intragranular ingredient mixture to form the core of the FDC; and coating the core in a coat until the weight of the coat is 1-5% of the weight of the FDC.

In some embodiments, the present disclosure provides a method of preparing an FDC of Drotaverine or a pharmaceutically acceptable salt thereof and Chlordiazepoxide or a pharmaceutically acceptable salt thereof comprising a core with a coating using a dry granulation process, the method comprising: Mixing intragranular ingredients for the core of the FDC; slugging or compacting the intragranular ingredient mixture to obtain compacts; milling and sieving the compacts to obtain sieved granules; mixing the sieved granules with at least one excipient to obtain a final blend; loading the final blend on a tableting machine; compressing the final blend to form the core of the FDC; and coating the core in a coat until the weight of the coat is 1-5% of the weight of the FDC.

In some embodiments, the present disclosure provides a method of preparing an FDC of Drotaverine or a pharmaceutically acceptable salt thereof and Chlordiazepoxide or a pharmaceutically acceptable salt thereof comprising a core with a coating using a wet granulation process, the method comprising: Mixing intragranular ingredients for the core of the FDC; shearing the intragranular ingredient mixture along with a binder solution using a rapid mixer granulator to obtain granules; drying the granules to obtain dried granules; mixing the dried granules with at least one excipient to obtain a final blend; loading the final blend on a tableting machine; compressing the final blend to form the core of the tablet; and coating the core in a coat until the weight of the coat is 1-5% of the weight of the FDC.

In some embodiments, the present disclosure provides a method of preparing an FDC of Drotaverine or a pharmaceutically acceptable salt thereof and Chlordiazepoxide or a pharmaceutically acceptable salt thereof comprising a core with a coating using a fluidized bed process, the method comprising: Mixing intragranular ingredients for a core of the FDC; forming granules from the mixed intragranular ingredients and a binder solution by fluidized bed granulation; drying the granules to obtain dried granules; mixing the dried granules with at least one excipient to obtain a final blend; loading the final blend on a tableting machine; compressing the final blend to form the core of the FDC; and coating the core in a coat until the weight of the coat is 1-5% of the weight of the FDC.

In some embodiments, the present disclosure provides a method of preparing an FDC of Drotaverine or a pharmaceutically acceptable salt thereof and Chlordiazepoxide or a pharmaceutically acceptable salt thereof comprising a multi-layer core with a coating using a direct compression process, the method comprising: Mixing intragranular ingredients for each layer of the core separately; loading the separate final blends for each layer of the core on a tableting machine; compressing the separate final blends to form the multi-layer core of the FDC; and coating the core in a coat until the weight of the coat is 1-5% of the weight of the fixed dose combination.

In some embodiments, the present disclosure provides a method of preparing an FDC of Drotaverine or a pharmaceutically acceptable salt thereof and Chlordiazepoxide or a pharmaceutically acceptable salt thereof comprising a multi-layer core with a coating using a dry granulation process, the method comprising: Mixing intragranular ingredients for each layer of the core; slugging or compacting the separate intragranular ingredient mixtures to obtain compacts for each layer of the core; milling and sieving the separate compacts to obtain sieved granules for each layer of the core; mixing the separate sieved granules with at least one excipient to obtain a final blend for each layer of the core; loading the separate final blends for each layer of the core on a tableting machine; compressing the separate final blends to form the multi-layer core of the FDC; and coating the core in a coat until the weight of the coat is 1-5% of the weight of the FDC.

In some embodiments, the present disclosure provides a method of preparing an FDC of Drotaverine or a pharmaceutically acceptable salt thereof and Chlordiazepoxide or a pharmaceutically acceptable salt thereof comprising a multi-layer core with a coating using a wet granulation process, the method comprising: Mixing intragranular ingredients for each layer of the multi-layer core; shearing the separate intragranular ingredient mixtures along with a binder solution using a rapid mixer granulator to obtain granules for each layer of the core; drying the separate granules to obtain dried granules for each layer of the core; mixing the separate dried granules with at least one excipient to obtain a final blend for each layer of the core; loading the separate final blends for each layer of the core on a tableting machine; compressing the final blends to form the multi-layer core of the FDC; and coating the core in a coat until the weight of the coat is 1-5% of the weight of the FDC.

In some embodiments, the present disclosure provides a method of preparing an FDC of Drotaverine or a pharmaceutically acceptable salt thereof and Chlordiazepoxide or a pharmaceutically acceptable salt thereof comprising a multi-layer core with a coating using a fluidized bed process, the method comprising: Mixing intragranular ingredients for each layer of the core; forming granules from the mixed intragranular ingredients and a binder solution by fluidized bed granulation; drying the separate granules to obtain dried granules for each layer of the core; mixing the separate dried granules with at least one excipient to obtain a final blend for each layer of the core; loading the separate final blends for each layer of the core on a tableting machine; compressing the final blends to form the multi-layer core of the FDC; and coating the core in a coat until the weight of the coat is 1-5% of the weight of the FDC.

In some embodiments, the present disclosure provides a method of treating at least one symptom of gastrointestinal disorder characterized by spastic conditions of smooth muscles in a subject in need thereof. Certain exemplary conditions that can be treated according to some embodiments include treatment of irritable bowel syndrome (irritable colon, spastic colon, mucous colitis), treatment of gastrointestinal manifestations of anxiety and tension in the gastrointestinal tract, treatment of abdominal pain, or treatment of gastrointestinal spasms secondary to organic diseases of the digestive system such as gastric and duodenal ulcers, acute enterocolitis, etc.

In some embodiments, the present disclosure provides a pharmaceutical composition for use in treating at least one symptom of gastrointestinal disorder characterized by spastic conditions of smooth muscles in a subject in need thereof. In some embodiments, the pharmaceutical composition is used in the treatment of irritable bowel syndrome (irritable colon, spastic colon, mucous colitis), treatment of gastrointestinal manifestations of anxiety and tension in the gastrointestinal tract, treatment of abdominal pain, or treatment of gastrointestinal spasms secondary to organic diseases of the digestive system such as gastric and duodenal ulcers, acute enterocolitis, or a combination thereof.

In some embodiments, the present disclosure provides a combination comprising Drotaverine or a pharmaceutically acceptable salt thereof and at least one benzodiazepine formulated as a single medicine.

### DETAILED DESCRIPTION

In some aspects, the present disclosure relates to a pharmaceutical FDC of antispasmodic and anxiolytic agents and/or one or more pharmaceutically acceptable excipients. In some embodiments, the combination is an FDC suitable for oral route of administration. In some embodiments, the disclosure relates to chemically stable pharmaceutical formulations including Drotaverine and Chlordiazepoxide or pharmaceutically acceptable salts thereof and one or more pharmaceutically acceptable excipients and uses thereof.

In some embodiments, the disclosure discusses novel stable FDCs. In some embodiments, the novel pharmaceutical compositions are chemically stable and bioavailable FDC products. Based on accelerated stability data, certain FDC formulations have significantly reduced chemical degradation when formulated as an FDC of the active agents in a single-layer core, a bi-layered core, or a multi-layered core. In some embodiments, the dosage forms are ‘Tablet in Tablet’ or ‘Compression coated Tablets’, ‘Tablet in Capsules,’ ‘multi-layered tablets,’ drug coated pellets,' or ‘mini-tablets filled in capsules.’ In some embodiments, the dosage forms are compressed or coated Tablet in hard capsules, multi-layered tablets, compression coated tablets. In some embodiments, the dosage forms do not have a coating. In some embodiments, the tablets have a coating.

In some embodiments, the present disclosure provides an FDC comprising anti-anxiety agent and anti-spasmodic agent and one or more pharmaceutically acceptable excipients.

In some embodiments, the present disclosure relates to an FDC comprising Chlordiazepoxide, Drotaverine, and one or more pharmaceutically acceptable excipients.

In some embodiments, the present disclosure relates to an FDC comprising Chlordiazepoxide, Drotaverine, and one or more pharmaceutically acceptable excipients comprising fillers, binders, stabilizers, antioxidant, disintegrants, lubricants, or coating materials, or combinations thereof.

In some embodiments, the present disclosure relates to an FDC comprising Chlordiazepoxide, Drotaverine, and pharmaceutically acceptable excipients which include an acidifier.

In some embodiments, the present disclosure relates to an FDC comprising Chlordiazepoxide, Drotaverine, and pharmaceutically acceptable excipients which include an acidifier comprising citric acid, fumaric acid, lactic acid, maleic acid, malic acid, or tartaric acid, or combinations thereof.

In some embodiments, the present disclosure relates to an FDC comprising Chlordiazepoxide, Drotaverine, and pharmaceutically acceptable excipients which include an antioxidant.

In some embodiments, the present disclosure relates to an FDC comprising Chlordiazepoxide, Drotaverine, and pharmaceutically acceptable excipients which include an antioxidant comprising butylated hydroxy-anisole (BHA), butylated hydroxytoluene (BHT), sodium metabisulfite, sodium thiosulfate, propyl gallate, ascorbic acid, or cysteine, or combinations thereof.

In some embodiments, the present disclosure relates to an 1-DC of Chlordiazepoxide and Drotaverine, and one or more pharmaceutically acceptable excipients, wherein the pharmaceutical formulation comprises a core with one or more layers and a coating surrounding the core and is used in the treatment of the irritable bowel syndrome (irritable colon, spastic colon, mucous colitis), treatment of GI manifestations of anxiety and tension in the gastrointestinal tract, treatment of abdominal pain, or treatment gastrointestinal spasms secondary to organic diseases of the digestive system, such as gastric and duodenal ulcers, acute enterocolitis, etc.

In some embodiments, the present disclosure relates to an FDC comprising Chlordiazepoxide, Drotaverine, and one or more pharmaceutically acceptable excipients, wherein the FDC comprises a core with one or more layers and a coating surrounding the core, wherein the coating comprises a gelatin shell or non-gelatin based material, such as HPMC or plant materials.

In some embodiments, the coating comprises a coating agent that comprises polyvinylpyrrolidone (PVP), methyl cellulose, ethyl cellulose, hydroxypropyl cellulose (HPC), hydroxypropylmethyl cellulose (HPMC), Sodium Carboxymethylcellulose, dextrin, maltodextrin, lactose, D-mannitol, polyvinyl alcohol polymer, methacrylic acid copolymer, aminoalkylmethacrylate copolymer, or ethyl acrylate methyl methacrylate copolymer, or combinations thereof.

In some embodiments, the coating agent comprises a plasticizer (for example, Triacetin, Diethyl phthalate etc), an anti-tacking agent (for example, Talc), an opacifier (for example, Titanium or Aluminium oxides), or a colouring agent (for example, natural and synthetic colours approved for pharmaceutical use), or combinations thereof.

In some embodiments, film-coated formulations comprise one or more film coating base agents comprising polyvinyl alcohol or sodium carboxymethyl cellulose, or methacrylic acid copolymer, or combinations thereof. Certain of these embodiments have shown improved storage stability for both Drotaverine and Chlordiazepoxide actives.

In some embodiments, the solvent system used for film coating comprises a water-based solvent. In some embodiments, the solvent system comprises a hydroalcoholic solvent. In some embodiments, the solvent system comprises a non-aqueous solvent comprising Methylene chloride, Acetone, or Isopropyl alcohol, or combinations thereof.

In some embodiments, the disclosed FDCs use packaging forms that suppress moisture permeation. Examples of such suitable packaging material include a high-density polyethylene (HDPE) bulk dosage unit container. In some embodiments, this container also may contain a desiccant. A desiccant is any drying agent that removes moisture from the air. Certain exemplary desiccants include silica gel, clay desiccants, calcium sulfate, calcium chloride, calcium oxide, zeolite, activated alumina, activated charcoal, and combinations thereof.

In some embodiments, the disclosed FDCs of Drotaverine and Chlordiazepoxide are packaged in unit dose packaging which are substantially impermeable to gas exchange, such as Aluminium/Aluminium (Alu/Alu) blisters, an Alu-polychloro-3-floroethylene himopolymer/PVC Laminate blister.

In some embodiments of the present disclosure, the FDCs demonstrate good chemical stability throughout the shelf life of drug product and comply with standards desired by regulatory agencies worldwide. In some embodiments, the FDCs are formulated into a dosage form such as rapid release, immediate-release, slow-release, sublingual, intravenous, controlled release, delayed-release, a combination of immediate and controlled release, nano-encapsulation formulations, and/or a tamper-resistant or abuse-resistant delivery system.

In some embodiments, the FDCs disclosed herein comprise a benzodiazepine. Benzodiazepines are anti-anxiety medications used to treat anxiety disorders. In some embodiments, the anxiolytic agent is a benzodiazepine. Exemplary benzodiazepines include Clonazepam, Alprazolam, Diazepam, Oxazepam, Lorazepam, or Chlordiazepoxide, or a combination thereof.

The inventors' initial lab studies showed that Chlordiazepoxide API shows significant degradation in acidic environment, as well as in basic and oxidative conditions. While Drotaverine HCl exhibited best chemical stability in acidic pH, it degrades significantly in basic and peroxide solution. Therefore, drug-interaction studies were undertaken by the inventors and the samples were evaluated for chemical stability. It was observed that when Chlordiazepoxide and Drotaverine HCl APIs were combined together, it resulted in significant chemical interaction between the two active ingredients. There is significant degradation of Chlordiazepoxide and Drotaverine when they were mixed together. Drotaverine and Chlordiazepoxide active drugs in a combination or formulation undergo chemical interaction thereby resulting in degradation of the active moieties and formation of degradants which are not therapeutically active as shown in Table 1 below. The presence of significant amounts of impurities indicated that mixing of Drotaverine and Chlordiazepoxide results in mutual degradation.

TABLE 1
Impurities obtained after mixing Drotaverine HCL and
Chlordiazepoxide API

| | Condition | | | |
|---|---|---|---|---|
| | | | 40° C./75% RH | |
| | Initial | | (1 month) | |
| Name of Impurity | RRT | % w/w | RRT | % w/w |
| Unknown in lieu of | 0.89 | 0.67 | 0.89 | 25.16 |
| Chlordiazepoxide active | | | | |
| moiety | | | | |
| Unknown in lieu of | 1.65 | 0.00 | 1.65 | 0.17 |
| Drotaverine active moiety | | | | |
| Other Unknown | 1.51 | 0.00 | 1.51 | 0.48 |
| Total Impurities | | 0.67 | | 25.81 |

In some embodiments, the present disclosure provides stable pharmaceutical fixed dose combinations (FDCs) delivering Drotaverine and Chlordiazepoxide in a single pill for ease of administration and better patient compliance. In some embodiments, the FDCs are immediate release dosage forms of Drotaverine and Chlordiazepoxide, which provides equivalent or similar performance to standalone marketed products of Drotaverine tablets and Chlordiazepoxide tablets. In some embodiments, the FDCs also exhibit one or more of a sustained release, controlled-release, and immediate-release dissolution profile. In some embodiments, the disclosure provides single-layer cores or single-layer core drug release systems. For example, in some embodiments the FDC is formulated as a single-layer core with a coating or immediate release capsules. In some embodiments, the disclosure provides ‘multi-layered’ or ‘multi-component’ dosage form units. In some embodiments, the dosage forms are ‘Tablet in Tablet’ or ‘Compression coated Tablets,’ Tablet in Capsules,' multi-layered tablets,' drug coated pellets,' or ‘mini-tablets filled in capsules.’ For example, in some embodiments the FDC is formulated as a bi-layer or multi-layer core with a coating. In some embodiments, the disclosure provides chemically stable FDCs of Drotaverine and Chlordiazepoxide to minimize the significant chemical interaction of these two drugs when mixed together.

In some embodiments, the FDCs are manufactured by direct compression, dry granulation, wet granulation, or fluidized bed processing techniques. In some embodiments, the Drotaverine and Chlordiazepoxide or their pharmaceutically acceptable salts are physically separated from one another. In some embodiments, the physical separation includes placing a separating layer or barrier layer “sandwiched” between the layer containing Drotaverine and the layer containing Chlordiazepoxide. In some embodiments, the Drotaverine and Chlordiazepoxide are separated by including one of the actives in a core and the other active in a coating. In some embodiments, the Drotaverine and Chlordiazepoxide are separated by including the actives in granulation or pellets, containing one of the actives. In some embodiments, coating one or more of the API-containing compositions is employed and the dosage forms can then be formulated as tablets or capsules.

In some embodiments, the disclosure addresses the problem of instability due to chemical interaction of Drotaverine and Chlordiazepoxide or their pharmaceutically acceptable salts by way of novel formulation designs along with at least one acidic component or antioxidant in the FDC. In some embodiments, the physical separation includes placing a separating layer or barrier layer “sandwiched” between the layer containing Drotaverine and the layer containing Chlordiazepoxide. In some embodiments, the Drotaverine and Chlordiazepoxide are separated by including one of the actives in a core and the other active in a coating. In some embodiments, the Drotaverine and Chlordiazepoxide are separated by including the actives in granulation or pellets, containing one of the actives. In some embodiments, coating one or more of the API-containing compositions is employed and the dosage forms can then be formulated as tablets or capsules.

In some embodiments, the FDCs are prepared by direct compression comprising blending Drotaverine along with the pharmaceutically acceptable excipients in a core, and coating the core with a coating comprising Chlordiazepoxide, with or without an inactive polymer layer between the core and Chlordiazepoxide coating. In some embodiments, the steps involved in the direct compression process are as follows:

Weighing and mixing of intragranular ingredients including active drug, diluents, disintegrants, and lubricants of the formulation to obtain a final blend. The final blend is loaded on tabletting machine which is followed by compression to form a core. The compressed core is then coated using suitable film coating compositions until the weight of the coat is 1-5% of the weight of the FDC.

In some embodiments, an FDC is prepared by dry granulation of Drotaverine along with the pharmaceutically acceptable excipients in a core, while Chlordiazepoxide is coated over the core containing Drotaverine with or without inactive polymer layer between the core and Chlordiazepoxide coating. In some embodiments, the steps involved in the dry granulation process are as follows:

Weighing and mixing of intragranular ingredients of the formulation is performed; followed by slugging or compaction of the mixed ingredients to obtain compacts. After compaction, milling and sieving of the compacts is undertaken to obtain sieved granules. The sieved granules are thereafter mixed with disintegrants and lubricants resulting in formation of final blend. The final blend is loaded on tabletting machine which is followed by compression to form the core. The compressed core can be coated using suitable film coating compositions until the weight of the coat is 1-5% of the weight of the FDC.

In some embodiments, an FDC is prepared by wet granulation comprising Drotaverine and pharmaceutically acceptable excipients in a core, while Chlordiazepoxide is coated over the core containing Drotaverine with or without inactive polymer layer between the Drotaverine core and Chlordiazepoxide coating. In some embodiments, the steps involved in the wet granulation process are as follows:

Weighing and mixing of intragranular ingredients of the formulation to obtain mixed granules is performed by high shear granulation along with a binder solution using a rapid mixer granulator (SARAL Engineering, Gujarat, India). The mixing is followed by drying to obtain dried granules. The dried granules are thereafter mixed with disintegrants and lubricants resulting in final blend for the core. The final blend is loaded on a tabletting machine followed by compression to obtain a compressed tablet. The compressed tablets can be coated using suitable film coating compositions until the weight of the coat is 1-5% of the weight of the FDC.

In some embodiments, an FDC is prepared by fluidized bed processing involving preparing Drotaverine granules along with pharmaceutically acceptable excipients in a core, while Chlordiazepoxide is coated over the core with or without inactive layer between the core and Chlordiazepoxide coating. In some embodiments, the steps involved in the fluidized bed processing process are as follows:

Weighing and mixing of intragranular ingredients of the formulation, followed by forming granules from the mixed intragranular ingredients and a binder solution by fluidized bed granulation; followed by drying of the granules to obtain dried granules. The dried granules are thereafter mixed with disintegrants and lubricants resulting in formation of final blend. The final blend is loaded on tabletting machine and is compressed to form the core. The compressed core can be coated using suitable film coating compositions until the weight of the coat is 1-5% of the weight of the FDC.

In some embodiments, an FDC is prepared by direct compression involving preparing individual core layers, one layer comprising Chlordiazepoxide and pharmaceutically acceptable excipients and another layer comprising Drotaverine and pharmaceutically acceptable excipients. In some embodiments, the individual layers' blend are then compressed to form the bilayer core. In some embodiments, a tri-layer core is formed by including an inert excipient layer sandwiched between the Drotaverine layer and the Chlordiazepoxide layer. In some embodiments, the inert excipient comprises Mannitol, Dicalcium Phosphate, Lactose anhydrous, Colloidal Silicon dioxide, Starch (dried), Magnesium stearate, or Purified Talc. In some embodiments, the steps involved in the direct compression process are as follows:

For each layer, separately weighing and mixing of intragranular ingredients of the formulation including active drug, diluents, disintegrants, and lubricants to obtain separate final blends of the individual layers of the bilayer core. The final blend of the two layers is loaded in separate hoppers on a tabletting machine and compressed to obtain the compressed bilayer core. The compressed bilayer core can be coated using suitable film coating compositions until the weight of the coat is 1-5% of the weight of the FDC.

In some embodiments, an FDC is prepared by dry granulation involving preparing individual core layers, one core layer comprising Chlordiazepoxide and pharmaceutically acceptable excipients and another layer comprising Drotaverine and pharmaceutically acceptable excipients. In some embodiments, the individual layers are then compressed to form a bilayer core. In some embodiment, a tri-layer core is formed by including an inert excipient layer sandwiched between the Drotaverine layer and the Chlordiazepoxide layer. In some embodiments, the inert excipient comprises Mannitol, Dicalcium Phosphate, Lactose anhydrous, Colloidal Silicon dioxide, Starch (dried), Magnesium stearate, or Purified Talc. In some embodiments, the manufacturing steps involved in the dry granulation process are as follows:

Weighing and mixing of intragranular ingredients of the formulation to obtain mixed ingredients for each layer of the core separately; followed by slugging or compaction of the separate mixed ingredients to obtain compacts for each layer of the core. After compaction, milling and sieving of the separate compacts is performed for each layer of the core. The separate sieved granules are thereafter mixed with disintegrants and lubricants resulting in formation of the final blend of the two separate layers. The final blend of each individual layer is loaded in separate hoppers on a tabletting machine followed by compression to form the compressed bilayer core. The compressed bilayer core can be coated using suitable film coating compositions until the weight of the coat is 1-5% of the weight of the FDC.

In some embodiments, an FDC is prepared by wet granulation involving preparing individual core layers, one layer comprising Chlordiazepoxide and pharmaceutically acceptable excipients and another layer comprising Drotaverine and pharmaceutically acceptable excipients. In some embodiments, the individual layers are then compressed to form a bilayer core. In some embodiments, a tri-layer core is obtained by including an inert excipient layer sandwiched between the Drotaverine layer and Chlordiazepoxide layer. In some embodiments, the inert excipient comprises Mannitol, Dicalcium Phosphate, Lactose anhydrous, Colloidal Silicon dioxide, Starch (dried), Magnesium stearate, or Purified Talc. In some embodiments, the steps involved in the wet granulation process are as follows:

Weighing and mixing of intragranular ingredients of the formulation for each layer of the core separately, shearing the mixed intragranular ingredients along with a binder solution to obtain mixed granules for each layer of the core by high shear granulation using a rapid mixer granulator (SARAL Engineering, Gujarat, India). The separate granules and then dried to obtain dried granules for each layer of the core. The separate dried granules are thereafter mixed with disintegrants and lubricants resulting in separate final blends of the individual layers. The separate final blends of the separate layers are loaded in separate hoppers on a tabletting machine followed by compression to obtain a compressed bilayer core. The compressed tablets can be coated using suitable film coating compositions until the weight of the coat is 1-5% of the weight of the FDC.

In some embodiments, an FDC is prepared by fluidized bed processing involving preparing individual core layers, one layer comprising Chlordiazepoxide and pharmaceutically acceptable excipients and another layer comprising Drotaverine and pharmaceutically acceptable excipients. In some embodiments, the individual layers are then compressed to form a bilayer core. In some embodiments, a tri-layer core is obtained by including an inert excipient layer sandwiched between the Drotaverine layer and Chlordiazepoxide layer. In some embodiments, the inert excipient comprises Mannitol, Dicalcium Phosphate, Lactose anhydrous, Colloidal Silicon dioxide, Starch (dried), Magnesium stearate, or Purified Talc. In certain embodiments, the steps involved in the fluidized bed processing process are as follows:

Weighing and mixing of intragranular ingredients of the formulation for each layer of the core separately, followed by forming granules for each layer of the core from the mixed intragranular ingredients and a binder solution by fluidized bed granulation. The separate granules are then dried to obtain dried granules for each layer of the core. The separate dried granules are thereafter mixed with disintegrants and lubricants resulting in formation of separate final blends of the individual layers. The separate final blends of the separate layers is loaded in separate hoppers on a tabletting machine followed by compression to obtain a bilayer core. The compressed tablets can be coated using suitable film coating compositions until the weight of the coat is 1-5% of the weight of the FDC.

In some embodiments, an FDC is prepared using more than one process. In some embodiments, one process is used to prepare one layer of the FDC and a different process is used to prepare a second layer of the FDC. For example, one layer comprising Drotaverine or a pharmaceutically acceptable salt thereof is prepared using a direct compression process, a dry granulation process, a wet granulation process, or a fluidized bed process, and a second layer comprising a benzodiazepine is prepared using one of the other three processes. For example, one layer comprising Drotaverine or a pharmaceutically acceptable salt thereof is prepared using a wet granulation process and a second layer comprising a benzodiazepine is prepared using a direct compression process.

In some embodiments, a granulation liquid can comprise water or non-aqueous solvent along with other excipients such as diluents or filler, binders, surfactants, antioxidants, acidifiers, disintegrants, glidants, or lubricants, or combinations thereof.

In some embodiments, diluents used in the present disclosure include sugars such as lactose monohydrate, lactose anhydrous, mannitol; starches such as maize starch, corn starch, pregelatinized starches and starch 1500; and cellulose derivatives such as crystalline cellulose and powdered cellulose and directly compressible grade celluloses, In some embodiments, diluents include sorbitol, xylitol, calcium carbonate, magnesium carbonate, dibasic calcium phosphate, or tribasic calcium phosphate, or combinations thereof.

In some embodiments, disintegrants used in the present disclosure include dried starch, sodium starch glycollate, risperidone, croscarmellose sodium, L-HPC, or alginic acid, or combinations thereof.

In some embodiments, binders used in the present disclosure include hydroxypropyl cellulose, also called HPC (e.g., Klucel™ LF and Klucel™ EXF), various grades of hydroxypropyl methylcellulose, also called Hypromellose (e.g., Methocel™ products), various grades, polyvinylpyrrolidone or povidone (such as grades K25, K29, K30, and K90), copovidone (e.g., Plasdone™630), powdered acacia, gelatin, guar gum, carbomers (e.g., Carbopol® products), methylcellulose, polymethacrylates, or starches, or combinations thereof.

In some embodiments, surfactants used in the present disclosure include polyoxyethylene-polyoxypropylene block copolymers (poloxamers), alkyl sulfates (e.g., sodium lauryl sulfate sodium stearyl sulfate sodium oleyl sulfate and sodium cetyl sulfate), alkyl aryl sulfonate (e.g., sodium dodecyl benzene sulfonate and dialkyl sodium sulfosuccinate), polyethylene glycol, or polysorbates, or combinations thereof.

In some embodiments, lubricants used in the present disclosure include magnesium stearate, glyceryl monostearate, palmitic acid, talc, carnauba wax, calcium stearate, sodium stearate, sodium lauryl sulfate, magnesium lauryl sulfate, zinc stearate, polyoxymethylene monostearate, calcium silicate, silicon dioxide, hydrogenated vegetable oils and fats, stearic acid, or glyceryl behenate, or combinations thereof.

In some embodiments, glidants used in the present disclosure include colloidal silicon dioxide and/or talc.

In some embodiments, tablet dosage forms are film coated to mask bitter taste of Drotaverine while swallowing by patients. In some embodiments, the film coating of tablets also improved the storage stability of the drug product for both Drotaverine and Chlordiazepoxide actives.

A few examples for representative purpose without limiting scope of the disclosure are illustrated below.

### EXAMPLE 1

### Tablet of Drotaverine and Chlordiazepoxide (Drotaverine in the Core of the Tablet with Chlordiazepoxide in the Coating)

Tablets are made that comprise Drotaverine in the core and Chlordiazepoxide in film coating. The tablets are formulated by wet granulation process. The process comprises mixing of intragranular ingredients comprising Drotaverine (about 25 to 40% w/w) granules along with the pharmaceutically acceptable excipients in the core. The granules are dried and thereafter mixed with disintegrants and lubricants to form a final blend. The final blend is then loaded on a tableting machine and compressed to form a compressed core. The compressed core is coated using suitable film coating compositions. The coating over the core comprises Chlordiazepoxide (0.5 to 3.00% w/w) with or without inactive polymer coating in between the core and the Chlordiazepoxide coating layer.

The actives and pharmaceutically acceptable excipients in the tablets in % w/w are illustrated in Table 2 below:

TABLE 2
Actives and pharmaceutically acceptable excipients in
tablets comprising Drotaverine in the core and
Chlordiazepoxide in the film coating

| | | |
|---|---|---|
| Sr. No. | Ingredients | % w/w |

Mono layer tablet
Intra granular Material

| | | |
|---|---|---|
| 1 | Drotaverine HCl | 32.00 |
| 2 | Microcrystalline | 8.00 |
| | Cellulose | |
| 3 | Lactose Monohydrate | 35.00 |
| 4. | Citric acid | 5.00 |

Binder Solution

| | | |
|---|---|---|
| 5 | Purified water | q. s |
| 6 | PVP K 30 | 3.20 |

Extra granular Material

| | | |
|---|---|---|
| 7 | MCC PH 102 | 9.60 |
| 8 | Talc | 1.20 |
| 9 | Ascorbic acid | 2.00 |
| 10 | Magnesium Stearate | 2.00 |

Film Coating

| | | |
|---|---|---|
| 9 | Chlordiazepoxide | 2.00 |
| 10 | Film coating | 4.00 |
| | Formulation | |
| 11 | Purified Water | q.s. |
| 12 | Isopropyl Alcohol | q.s. |

### EXAMPLE 2

### Tablet of Drotaverine and Chlordiazepoxide (Drotaverine and Chlordiazepoxide in the Core of the tablet)

Tablets are made that comprise Drotaverine and Chlordiazepoxide in the core, and which further contain a film coating. The tablets are formulated by wet granulation method. The process comprises mixing of intragranular ingredients comprising Drotaverine (about 25 to 40% w/w) and Chlordiazepoxide (about 0.5 to 3.5% w/w) granules along with the pharmaceutically acceptable excipients in the core. The granulation method, and the excipients used in the method, stabilize the Drotaverine HCl and Chlordiazepoxide HCl in the tablet. The granules are dried and thereafter mixed with disintegrants and lubricants to form the final blend. The final blend is then loaded on a tableting machine and compressed to form a compressed core. The compressed core is coated using suitable film coating compositions.

The actives and pharmaceutically acceptable excipients in the tablets in % w/w are illustrated in Table 3 below:

TABLE 3
Actives and pharmaceutically acceptable excipients in
monolayer tablets comprising Drotaverine and
Chlordiazepoxide in the core

| | | |
|---|---|---|
| Sr. No. | Ingredients | % w/w |

Mono layer tablet
Intra granular Material

| | | |
|---|---|---|
| 1 | Drotaverine HCl | 32.00 |
| | Chlordiazepoxide HCl | 2.24 |
| 2 | Maize Starch | 8.00 |
| 3 | Lactose Monohydrate | 36.76 |
| 4 | Maleic acid | 3.00 |

Binder Solution

| | | |
|---|---|---|
| 5 | IPA | q.s. |
| 6 | PVP K 30 | 3.20 |

Extra granular Material

| | | |
|---|---|---|
| 7 | MCC PH 102 | 11.60 |
| 8 | Talc | 1.20 |
| 9 | Sodium Stearyl Fumarate | 2.00 |

Film Coating

| | | |
|---|---|---|
| 10 | Film coating formulation | 4.00 |
| 11 | Purified Water | q.s. |

### EXAMPLE 3

### Bi-Layer Tablets of Drotaverine and Chlordiazepoxide (Wet Granulation Process)

Bi-layer tablets are made that comprise Drotaverine and Chlordiazepoxide in separate layers in the core. The tablets are formulated by wet granulation method.

This process of preparing bi-layer tablets comprising Drotaverine and Chlordiazepoxide in separate layers comprises separately mixing of the actives Drotaverine (about 30 to 50% w/w) and Chlordiazepoxide (about 2 to 7% w/w) and intragranular ingredients to obtain mixed granules by high shear granulation by using a rapid mixer granulator. The mixed granules are then subjected to drying to obtain dried granules. The dried granules are thereafter mixed with disintegrants and lubricants, resulting in the formation of a final blend for the Drotaverine layer and a final blend for the Chlordiazepoxide layer. The separate layers are loaded into separate hoppers on a tableting machine, followed by compression of the separate layers to obtain a compressed bilayer core. The compressed bilayer core is then coated using suitable film coating compositions.

The actives and pharmaceutically acceptable excipients in the tablets in % w/w are illustrated in Tables 4-7 below:

TABLE 4
Actives and pharmaceutically acceptable excipients in
bilayer tablets comprising Drotaverine and
Chlordiazepoxide in separate layers.

| Sr. No. | Ingredients | % w/w |
|---|---|---|
| 1st layer | | |
| Intra granular Material | | |
| 1 | Drotaverine HCl | 40.00 |
| 2 | Maize Starch | 5.00 |
| 3 | Lactose Monohydrate | 38.50 |
| 4 | Fumaric acid | 4.00 |

Binder Solution

| | | |
|---|---|---|
| 4 | IPA | q.s. |
| 5 | Hydroxypropyl Cellulose | 2.50 |

Extra granular Material

| 6 | Crospovidone | 7.50 |
|---|---|---|
| 7 | Talc | 1.00 |
| 8 | Stearic acid | 1.50 |
| 2nd layer | | |
| Intra granular Material | | |
| 1 | Chlordiazepoxide | 5.00 |
| 2 | Maize Starch | 10.00 |
| 3 | Lactose Monohydrate | 58.70 |
| 4 | Sunset yellow lake | 0.30 |

Binder Solution

| | | |
|---|---|---|
| 4 | IPA | q.s. |
| 5 | PVP K 30 | 3.00 |

Extra granular Material

| | | |
|---|---|---|
| 6 | MCC PH 102 | 18.00 |
| 7 | Sodium metabisulfite | 2.00 |
| 8 | Talc | 1.00 |
| 9 | Glyceryl di behenate | 2.00 |

Film Coating

| | | |
|---|---|---|
| 1 | Film Coating material | 2.50 |

TABLE 5
Actives and pharmaceutically acceptable excipients in
bilayer tablets comprising Drotaverine and
Chlordiazepoxide in separate layers

| Sr. No. | Ingredients | % w/w |
|---|---|---|
| 1st layer | | |
| Intra granular Material | | |
| 1 | Drotaverine HCl | 35.00 |
| 2 | Maize Starch | 5.00 |
| 3 | Lactose Monohydrate | 43.50 |
| 4 | Fumaric acid | 4.00 |

Binder Solution

| | | |
|---|---|---|
| 4 | IPA | q.s. |
| 5 | Povidone K-90 | 2.50 |

Extra granular Material

| 6 | Crospovidone | 7.50 |
|---|---|---|
| 7 | Talc | 1.00 |
| 8 | Stearic acid | 1.50 |
| 2nd layer | | |
| Intra granular Material | | |
| 1 | Chlordiazepoxide | 7.00 |
| 2 | Maize Starch | 10.00 |
| 3 | Lactose Monohydrate | 56.70 |
| 4 | Sunset yellow lake | 0.30 |

Binder Solution

| | | |
|---|---|---|
| 4 | IPA | q.s. |
| 5 | PVP K 30 | 3.00 |

Extra granular Material

| | | |
|---|---|---|
| 6 | MCC PH 102 | 18.00 |
| 7 | Sodium metabisulfite | 2.00 |
| 8 | Talc | 1.00 |
| 9 | Glyceryl di behenate | 2.00 |

Film Coating

| | | |
|---|---|---|
| 1 | Film Coating material | 2.50 |

TABLE 6
Actives and pharmaceutically acceptable excipients in
bilayer tablets comprising Drotaverine and
Chlordiazepoxide in separate layers

| Sr. No. | Ingredients | % w/w |
|---|---|---|
| 1st layer | | |
| Intra granular Material | | |
| 1 | Drotaverine HCl | 45.00 |
| 2 | Maize Starch | 6.00 |
| 3 | Lactose Monohydrate | 33.50 |
| 4 | Citric acid | 3.00 |

Binder Solution

| | | |
|---|---|---|
| 4 | IPA | q.s. |
| 5 | Hydroxypropylmethyl | 2.50 |
| | Cellulose | |

Extra granular Material

| 6 | Crospovidone | 7.50 |
|---|---|---|
| 7 | Talc | 1.00 |
| 8 | Stearic acid | 1.50 |
| 2nd layer | | |
| Intra granular Material | | |
| 1 | Chlordiazepoxide | 6.00 |
| 2 | Maize Starch | 10.00 |
| 3 | Lactose Monohydrate | 59.70 |
| 4 | Sunset yellow lake | 0.30 |

Binder Solution

| | | |
|---|---|---|
| 4 | IPA | q.s. |
| 5 | PVP K 30 | 3.00 |

Extra granular Material

| | | |
|---|---|---|
| 6 | MCC PH 102 | 18.00 |
| 7 | Sodium metabisulfite | 2.00 |
| 8 | Talc | 1.00 |
| 9 | Glyceryl di behenate | 2.00 |

Film Coating

| | | |
|---|---|---|
| 1 | Film Coating material | 2.50 |

TABLE 7
Actives and pharmaceutically acceptable excipients in
bilayer tablets comprising Drotaverine and
Chlordiazepoxide in separate layers

| Sr. No. | Ingredients | % w/w |
|---|---|---|
| 1st layer | | |
| Intra granular Material | | |
| 1 | Drotaverine HCl | 50.00 |
| 2 | Maize Starch | 5.00 |
| 3 | Microcrystalline Cellulose | 28.50 |
| 4 | Fumaric acid | 4.00 |

Binder Solution

| | | |
|---|---|---|
| 4 | IPA | q.s. |
| 5 | Hydroxypropyl Cellulose | 2.50 |

Extra granular Material

| 6 | Crospovidone | 7.50 |
|---|---|---|
| 7 | Talc | 1.00 |
| 8 | Stearic acid | 1.50 |
| 2nd layer | | |
| Intra granular Material | | |
| 1 | Chlordiazepoxide | 4.00 |
| 2 | Microcrystalline Cellulose | 11.00 |
| 3 | Lactose Monohydrate | 58.70 |
| 4 | Sunset yellow lake | 0.30 |

Binder Solution

| | | |
|---|---|---|
| 4 | IPA | q.s. |
| 5 | PVP K 30 | 3.00 |

Extra granular Material

| | | |
|---|---|---|
| 6 | MCC PH 102 | 18.00 |
| 7 | Sodium metabisulfite | 2.00 |
| 8 | Talc | 1.00 |
| 9 | Glyceryl di behenate | 2.00 |

Film Coating

| | | |
|---|---|---|
| 1 | Film Coating material | 2.50 |

### EXAMPLE 4

### Bi-Layer Tablets of Drotaverine and Chlordiazepoxide (Two Different Processes)

Bi-layer tablets are made that comprise Drotaverine and Chlordiazepoxide in separate layers in the core. The Drotaverine layer is prepared using a wet granulation method, while the Chlordiazepoxide layer is prepared using a direct compression method.

This process of preparing the Drotaverine layer comprises mixing Drotaverine (about 30 to 50% w/w) and intragranular ingredients to obtain mixed granules by high shear granulation along with a binder solution using a rapid mixer granulator. The mixed granules are then subjected to drying to obtain dried granules. The dried granules are thereafter mixed with disintegrants and lubricants, resulting in the formation of a final blend for the Drotaverine layer.

The process of preparing the Chlordiazepoxide layer comprises mixing Chlordiazepoxide (about 2 to 7% w/w) and the intragranular ingredients to obtain a final blend. The final blend is loaded on tabletting machine which is followed by compression to form a layer.

The Drotaverine layer and the Chlordiazepoxide layer are loaded into separate hoppers on a tableting machine, followed by compression of the separate layers to obtain a compressed bilayer core. The compressed bilayer core is then coated using suitable film coating compositions.

The actives and pharmaceutically acceptable excipients in the tablets in % w/w are illustrated in Table 8 below:

TABLE 8
Actives and pharmaceutically acceptable excipients in
bilayer tablets comprising Drotaverine and
Chlordiazepoxide in separate layers

| | | |
|---|---|---|
| Sr. No. | Ingredients | % w/w |

1st layer
Intragranular Material

| | | |
|---|---|---|
| 1 | Drotaverine HCl | 41.73 |
| 2 | MCC PH 200 | 20.00 |
| 3 | Mannitol | 21.77 |
| 4 | Croscarmellose sodium | 2.00 |

Binder Solution

| | | |
|---|---|---|
| 5 | Citric acid | 2.50 |
| 6 | PVP K 30 | 2.50 |
| 7 | IPA | q.s. |
| 8 | Water | q.s. |

Extragranular Material

| | | |
|---|---|---|
| 9 | Croscarmellose sodium | 2.00 |
| 10 | MCC PH 102 | 5.00 |
| 11 | Talc | 1.00 |
| 12 | Glyceryl di behenate | 1.00 |

2nd layer
Intragranular materials

| | | |
|---|---|---|
| 1 | Chlordiazepoxide | 5.02 |
| 2 | Silicified MCC | 48.98 |
| 3 | Mannitol | 40.00 |

Extragranular Materials

| | | |
|---|---|---|
| 4 | Croscaremellose sodium | 3.00 |
| 5 | Talc | 1.00 |
| 6 | Glyceryl di behenate | 2.00 |

Film coating

| | | |
|---|---|---|
| 1 | Film coating material | 3.00 |

### EXAMPLE 5

### Bi-Layer Tablets of Drotaverine and Chlordiazepoxide (Dry Granulation Process)

Bi-layer tablets are made that comprise Drotaverine and Chlordiazepoxide in separate layers in the core. The tablets are formulated by dry granulation method.

This process of preparing bi-layer tablets comprising Drotaverine and Chlordiazepoxide in separate layers comprises separately mixing of the actives Drotaverine (about 30 to 50% w/w) and Chlordiazepoxide (about 2 to 7% w/w) and the intragranular ingredients of each layer to obtain separate mixed ingredients for each layer of the core, followed by slugging or compaction of the separate mixed ingredients to obtain compacts for each layer of the core. After compaction, the separate compacts are milled and sieved to obtain separate sieved granules for each layer of the core. The separate sieved granules are thereafter mixed with disintegrants and lubricants resulting in formation of final blend of the two separate layers. The final blend of each individual layer is loaded in separate hoppers on a tabletting machine followed by compression to form the compressed bilayer core. The compressed bilayer core can be coated using suitable film coating compositions until the weight of the coat is 1-5% of the weight of the FDC.

The actives and pharmaceutically acceptable excipients in the tablets in % w/w are illustrated in Tables 9-11 below:

TABLE 9
Actives and pharmaceutically acceptable excipients in
bilayer tablets comprising Drotaverine and
Chlordiazepoxide in separate layers

| Sr. No. | Ingredients | % w/w |
|---|---|---|
| 1st layer | | |
| Intragranular Material | | |
| 1 | Drotaverine HCl | 41.73 |
| 2 | MCC PH 200 | 35.77 |
| 3 | Croscarmellose sodium | 8.00 |
| 4 | Citric acid | 2.50 |
| 5 | PVP K 30 | 2.50 |

Extragranular Material

| | | |
|---|---|---|
| 6 | Croscarmellose sodium | 2.00 |
| 7 | MCC PH 102 | 5.00 |
| 8 | Talc | 1.00 |
| 9 | Glyceryl di behenate | 1.50 |

Premix Material

| | | |
|---|---|---|
| 1 | Chlordiazepoxide | 5.02 |
| 2 | Silicified MCC | 48.98 |
| 3 | Mannitol | 40.00 |

Lubrication Material

| | | |
|---|---|---|
| 4 | Croscaremellose sodium | 3.00 |
| 5 | Talc | 1.00 |
| 6 | Glyceryl di behenate | 2.00 |

Film coating

| | | |
|---|---|---|
| 1 | Film coating material | 3.00 |

TABLE 10
Actives and pharmaceutically acceptable excipients in
bilayer tablets comprising Drotaverine and
Chlordiazepoxide in separate layers

| Sr. No. | Ingredients | % w/w |
|---|---|---|
| 1st layer | | |
| Intragranular Material | | |
| 1 | Drotaverine HCl | 41.73 |
| 2 | Dicalcium Phosphate | 31.77 |
| 3 | MCC PH101 | 12.00 |
| 4 | Citric acid | 2.50 |
| 5 | PVP K 30 | 2.50 |

Extragranular Material

| 6 | Croscarmellose sodium | 2.00 |
|---|---|---|
| 7 | MCC PH 102 | 5.00 |
| 8 | Talc | 1.00 |
| 9 | Glyceryl di behenate | 1.50 |
| 2nd layer | | |
| Premix Material | | |
| 1 | Chlordiazepoxide | 5.02 |
| 2 | Silicified MCC | 45.98 |
| 3 | Mannitol | 43.00 |

Lubrication Material

| | | |
|---|---|---|
| 4 | Croscaremellose sodium | 3.00 |
| 5 | Talc | 1.00 |
| 6 | Glyceryl di behenate | 2.00 |

Film coating

| | | |
|---|---|---|
| 1 | Film coating material | 3.00 |

TABLE 11
Actives and pharmaceutically acceptable excipients in
bilayer tablets comprising Drotaverine and
Chlordiazepoxide in separate layers

| Sr. No. | Ingredients | % w/w |
|---|---|---|
| 1st layer | | |
| Intragranular Material | | |
| 1 | Drotaverine HCl | 41.73 |
| 2 | Mannitol | 28.77 |
| 3 | MCC PH101 | 15.00 |
| 4 | Citric acid | 2.50 |
| 5 | PVP K 30 | 2.50 |

Extragranular Material

| 6 | Croscarmellose sodium | 2.00 |
|---|---|---|
| 7 | MCC PH 102 | 5.00 |
| 8 | Talc | 1.00 |
| 9 | Glyceryl di behenate | 1.50 |
| 2nd layer | | |
| Premix Material | | |
| 1 | Chlordiazepoxide | 5.02 |
| 2 | Dicalcium Phosphate | 42.98 |
| 3 | Mannitol | 46.00 |

Lubrication Material

| | | |
|---|---|---|
| 4 | Croscaremellose sodium | 3.00 |
| 5 | Talc | 1.00 |
| 6 | Glyceryl di behenate | 2.00 |

Film coating

| | | |
|---|---|---|
| 1 | Film coating material | 3.00 |

### EXAMPLE 4

### Stability Data

Certain FDCs discussed above were tested for stability. The FDCs showed good chemical stability as per currently available accelerated stability data using ICH guidelines (40° C.±2° C./75% RH±5% RH for 6 months). The stability data for exemplary monolayer and bilayer tablets of the FDC of the disclosure is presented in Table 12 and 13 below.

TABLE 12
Batch Stability Data for the FDC in Table 3

| Batch details | | | | |
|---|---|---|---|---|
| 40° C. ± 2° C./75% RH ± 5% RH in Alu-Alu Blister for Monolayer Tabs | | | | |
| Storage condition | | | | |
| | 1 M Acc | 2 M Acc | 3 M Acc | 6 M Acc |
| Initial | Alu/Alu | Alu/Alu | Alu/Alu | Alu/Alu |

Assay (Results)

| | | | | | |
|---|---|---|---|---|---|
| DROTAVERINE [Label Claim: 80.0 mg] | 99.65% | 99.53% | 98.37% | 98.21% | 98.20% |
| CHLORDIAZEPOXIDE [Label Claim: 5.0 mg] | 100.10% | 99.87% | 99.43% | 99.34% | 99.23% |

Related Substance (Results)

| | | | | | | |
|---|---|---|---|---|---|---|
| DROTAVERINE | DROTAVERINE ACID | ND | ND | ND | ND | ND |
| | DROTAVERINE AMINE | ND | ND | ND | ND | ND |
| | DROTAVERINE AMIDE | ND | ND | ND | ND | ND |
| | DROTAVERLADINE | 0.18 | 0.19 | 0.27 | 0.29 | 0.31 |
| | Highest unknown | 0.04 | 0.06 | 0..10 | 0.13 | 0.15 |
| CHLORDIAZEPOXIDE | CDO IMP-A | 0.05 | 0.24 | 0.34 | 0.43 | 0.58 |
| | CDO IMP-B | ND | ND | ND | ND | ND |
| | CDO IMP-C | ND | ND | ND | ND | ND |
| | Highest unknown | 0.03 | 0.05 | 0.08 | 0.08 | 0.09 |
| OTHERS | Other Highest Unknown | ND | 0.02 | 0.03 | 0.02 | 0.02 |

TABLE 13
Batch Stability Data for the FDC in Table 11

| Batch details | | | | |
|---|---|---|---|---|
| 40° C. ± 2° C./75% RH ± 5% RH in Alu-Alu Blister for Bilayer Tabs | | | | |
| Storage condition | | | | |
| | 1 M Acc | 2 M Acc | 3 M Acc | 6 M Acc |
| Initial | Alu/Alu | Alu/Alu | Alu/Alu | Alu/Alu |

Assay (Results)

| | | | | | |
|---|---|---|---|---|---|
| DROTAVERINE [Label Claim: 80.0 mg] | 100.10% | 99.85% | 98.67% | 98.53% | 98.23% |
| CHLORDIAZEPOXIDE [Label Claim: 5.0 mg] | 101.30% | 100.20% | 99.40% | 99.39% | 99.45% |

Related Substance (Results)

| | | | | | | |
|---|---|---|---|---|---|---|
| DROTAVERINE | DROTAVERINE ACID | ND | ND | ND | ND | ND |
| | DROTAVERINE AMINE | ND | ND | ND | ND | ND |
| | DROTAVERINE AMIDE | ND | ND | ND | ND | ND |
| | DROTAVERLADINE | 0.12 | 0.18 | 0.23 | 0.20 | 0.20 |
| | Highest unknown | 0.07 | 0.09 | 0.13 | 0.12 | 0.12 |
| CHLORDIAZEPOXIDE | CDO IMP-A | 0.01 | 0.21 | 0.38 | 0.49 | 0.51 |
| | CDO IMP-B | ND | ND | ND | ND | ND |
| | CDO IMP-C | ND | ND | ND | ND | ND |
| | Highest unknown | 0.02 | 0.04 | 0.06 | 0.06 | 0.08 |
| OTHERS | Other Highest Unknown | ND | 0.02 | 0.03 | 0.02 | 0.02 |

Although the subject matter has been described herein with reference to certain embodiments thereof, other embodiments are possible. For illustrative purpose, the combination and formulation of disclosure comprises Drotaverine and Chlordiazepoxide as the anti-spasmodic agent and anxiolytic agent, respectively. However, those skilled in the art would appreciate that scope of the disclosure would extend to other combinations and formulations of anti-spasmodic agents and anxiolytic agents known in the field of art.

What is claimed:

1. A fixed dose combination comprising Drotaverine or a pharmaceutically acceptable salt thereof and at least one benzodiazepine.

2. The fixed dose combination of claim 1, wherein the at least one benzodiazepine is Chlordiazepoxide or a pharmaceutically acceptable salt thereof.

3. The fixed dose combination of claim 1 or 2, wherein the fixed dose combination comprises a core, and wherein the core comprises at least one layer.

4. The fixed dose combination of any of claims 1-3, wherein the fixed dose combination comprises a coating surrounding the core.

5. The fixed dose combination of claim 4, wherein the core comprises one layer, and the core comprises Drotaverine or a pharmaceutically acceptable salt thereof and Chlordiazepoxide or a pharmaceutically acceptable salt thereof.

6. The fixed dose combination of claim 4, wherein the core comprises Drotaverine or a pharmaceutically acceptable salt thereof, and wherein the coating surrounding the core comprises Chlordiazepoxide or a pharmaceutically acceptable salt thereof.

7. The fixed dose combination of claim 4, wherein the core comprises at least two layers, and wherein one layer comprises Drotaverine or a pharmaceutically acceptable salt thereof, and wherein a second layer comprises Chlordiazepoxide or a pharmaceutically acceptable salt thereof.

8. The fixed dose combination of claim 7, wherein the core further comprises a third layer, wherein the third layer is between the one layer comprising Drotaverine or a pharmaceutically acceptable salt thereof and the second layer comprising Chlordiazepoxide or a pharmaceutically acceptable salt thereof, and wherein the third layer comprises an inert excipient.

9. The fixed dose combination of any of claims 1-8, wherein the fixed dose combination comprises 20-300 mg Drotaverine or a pharmaceutically acceptable salt thereof.

10. The fixed dose combination of any of claims 1-9, wherein the fixed dose combination comprises a pharmaceutically acceptable salt of Drotaverine, and wherein the pharmaceutically acceptable salt of Drotaverine is Drotaverine hydrochloride.

11. The fixed dose combination of any of claims 1-10, wherein the fixed dose combination comprises 2.5-40 mg Chlordiazepoxide or a pharmaceutically acceptable salt thereof.

12. The fixed dose combination of any of claims 1-11, wherein the fixed dose combination comprises a pharmaceutically acceptable salt of Chlordiazepoxide, and wherein the pharmaceutically acceptable salt of Chlordiazepoxide is Chlordiazepoxide hydrochloride.

13. The fixed dose combination of any of claims 1-12, wherein the coating further comprises a coating agent, wherein the coating agent comprises polyvinylpyrrolidone, methyl cellulose, ethyl cellulose, hydroxypropyl cellulose, hydroxypropylmethyl cellulose, sodium carboxymethylcellulose, dextrin, maltodextrin, lactose, D-mannitol, polyvinyl alcohol polymer, methacrylic acid copolymer, aminoalkylmethacrylate copolymer, or ethyl acrylate methyl methacrylate copolymer, or a combination thereof.

14. The fixed dose combination of claim 13, wherein the coating further comprises a plasticizer, an anti-tacking agent, an opacifier, or a coloring agent, or a combination thereof.

15. The fixed dose combination of any of claims 1-14, further comprising an acidifying agent, an antioxidant, a diluent, a binder, a surfactant, a lubricant, or a glidant, or a combination thereof.

16. The fixed dose combination of any of claims 1-15, wherein the acidifying agent comprises citric acid, fumaric acid, lactic acid, maleic acid, malic acid or tartaric acid, or a combination thereof.

17. The fixed dose combination of any of claims 1-16, wherein the fixed dose combination comprises an antioxidant, wherein the antioxidant comprises butylated hydroxy-anisole (BHA), butylated hydroxytoluene (BHT), sodium metabisulfite, sodium thiosulfate, propyl gallate, ascorbic acid, or cysteine, or a combination thereof.

18. The fixed dose combination of any of claims 1-17, wherein the fixed dose combination comprises a diluent, wherein the diluent comprises lactose monohydrate, lactose anhydrous, mannitol, maize starch, corn starch, pregelatinized starch, starch 1500, crystalline cellulose, powdered cellulose, directly compressible grade cellulose, sorbitol, xylitol, calcium carbonate, magnesium carbonate, dibasic calcium phosphate, or tribasic calcium phosphate, or a combination thereof.

19. The fixed dose combination of any of claims 1-18, wherein the fixed dose combination comprises a binder, wherein the binder comprises hydroxypropyl cellulose, hydroxypropyl methylcellulose, polyvinylpyrrolidone, copovidone, powdered acacia, gelatin, guar gum, a carbomer, methylcellulose, a polymethacrylate, or a starch, or a combination thereof.

20. The fixed dose combination of any of claims 1-19, wherein the fixed dose combination comprises a surfactant, wherein the surfactant comprises a polyoxyethylene-polyoxypropylene block copolymer, an alkyl sulfate, an alkyl aryl sulfonate, polyethylene glycol, or a polysorbate, or a combination thereof.

21. The fixed dose combination of any of claims 1-20, wherein the fixed dose combination comprises a lubricant, wherein the lubricant comprises magnesium stearate, glyceryl monostearate, palmitic acid, talc, carnauba wax, calcium stearate, sodium stearate, sodium lauryl sulfate, magnesium lauryl sulfate, zinc stearate, polyoxymethylene monostearate, calcium silicate, silicon dioxide, a hydrogenated vegetable oil or fat, stearic acid, or glyceryl behenate, or a combination thereof.

22. The fixed dose combination of any of claims 1-21, wherein the fixed dose combination comprises a glidant, wherein the glidant comprises colloidal silicon dioxide or talc, or a combination thereof.

23. The fixed dose combination of any of claims 1-22, wherein the fixed dose combination is prepared using a direct compression process.

24. The fixed dose combination of any of claims 1-22, wherein the fixed dose combination is prepared using a dry granulation process.

25. The fixed dose combination of any of claims 1-22, wherein the fixed dose combination is prepared using a wet granulation process.

26. The fixed dose combination of any of claims 1-22, wherein the fixed dose combination is prepared using a fluidized bed process.

27. The fixed dose combination of any of claims 1-26, wherein the fixed dose combination is packaged along with a pharmaceutically acceptable desiccant.

28. The fixed dose combination of any of claims 1-27, wherein the fixed dose combination is packaged in an inert gas environment.

29. A fixed dose combination comprising Drotaverine hydrochloride, Chlordiazepoxide, and Citric acid, wherein the formulation comprises a bilayer core with a coating, wherein Drotaverine hydrochloride is in a first layer of the core, and wherein Chlordiazepoxide is in a second layer in the core.

30. The fixed dose combination of claim 29, wherein the fixed dose combination is prepared using a dry granulation process.

31. A method of preparing a fixed dose combination of Drotaverine or a pharmaceutically acceptable salt thereof and Chlordiazepoxide or a pharmaceutically acceptable salt thereof comprising a core with a coating using a direct compression process, the method comprising:
a. mixing intragranular ingredients for the core of the fixed dose combination;
b. loading the intragranular ingredient on a tableting machine;
c. compressing the intragranular ingredient mixture to form the core of the fixed dose combination; and
d. coating the core in a coat until the weight of the coat is 1-5% of the weight of the fixed dose combination.

32. A method of preparing a fixed dose combination of Drotaverine or a pharmaceutically acceptable salt thereof and Chlordiazepoxide or a pharmaceutically acceptable salt thereof comprising a core with a coating using a dry granulation process, the method comprising:
a. mixing intragranular ingredients for the core of the fixed dose combination;
b. slugging or compacting the intragranular ingredient mixture to obtain compacts;
c. milling and sieving the compacts to obtain sieved granules;
d. mixing the sieved granules with at least one excipient to obtain a final blend;
e. loading the final blend on a tableting machine;
f. compressing the final blend to form the core of the fixed dose combination; and
g. coating the core in a coat until the weight of the coat is 1-5% of the weight of the fixed dose combination.

33. A method of preparing a fixed dose combination of Drotaverine or a pharmaceutically acceptable salt thereof and Chlordiazepoxide or a pharmaceutically acceptable salt thereof comprising a core with a coating using a wet granulation process, the method comprising:
a. mixing intragranular ingredients for the core of the fixed dose combination;
b. shearing the intragranular ingredient mixture along with a binder solution using a rapid mixer granulator to obtain granules;
c. drying the granules to obtain dried granules;
d. mixing the dried granules with at least one excipient to obtain a final blend;
e. loading the final blend on a tableting machine;
f. compressing the final blend to form the core of the tablet; and
g. coating the core in a coat until the weight of the coat is 1-5% of the weight of the fixed dose combination.

34. A method of preparing a fixed dose combination of Drotaverine or a pharmaceutically acceptable salt thereof and Chlordiazepoxide or a pharmaceutically acceptable salt thereof comprising a core with a coating using a fluidized bed process, the method comprising:
a. mixing intragranular ingredients for a core of the fixed dose combination;
b. forming granules from the mixed intragranular ingredients and a binder solution by fluidized bed granulation;
c. drying the granules to obtain dried granules;
d. mixing the dried granules with at least one excipient to obtain a final blend;
e. loading the final blend on a tableting machine;
f. compressing the final blend to form the core of the fixed dose combination; and
g. coating the core in a coat until the weight of the coat is 1-5% of the weight of the fixed dose combination.

35. A method of preparing a fixed dose combination of Drotaverine or a pharmaceutically acceptable salt thereof and Chlordiazepoxide or a pharmaceutically acceptable salt thereof comprising a multi-layer core with a coating using a direct compression process, the method comprising:
a. mixing intragranular ingredients for each layer of the core separately;
b. loading the separate final blends for each layer of the core on a tableting machine;
c. compressing the separate final blends to form the multi-layer core of the fixed dose combination; and
d. coating the core in a coat until the weight of the coat is 1-5% of the weight of the fixed dose combination.

36. A method of preparing a fixed dose combination of Drotaverine or a pharmaceutically acceptable salt thereof and Chlordiazepoxide or a pharmaceutically acceptable salt thereof comprising a multi-layer core with a coating using a dry granulation process, the method comprising:
a. mixing intragranular ingredients for each layer of the core separately;
b. slugging or compacting the separate intragranular ingredient mixtures to obtain compacts for each layer of the core;
c. milling and sieving the separate compacts to obtain separate sieved granules for each layer of the core;
d. mixing the separate sieved granules with at least one excipient to obtain a final blend for each layer of the core;
e. loading the separate final blends for each layer of the core on a tableting machine;
f. compressing the separate final blends to form the multi-layer core of the fixed dose combination; and
g. coating the core in a coat until the weight of the coat is 1-5% of the weight of the fixed dose combination.

37. A method of preparing a fixed dose combination of Drotaverine or a pharmaceutically acceptable salt thereof and Chlordiazepoxide or a pharmaceutically acceptable salt thereof comprising a multi-layer core with a coating using a wet granulation process, the method comprising:
a. mixing intragranular ingredients for each layer of the multi-layer core separately;
b. shearing the separate intragranular ingredient mixtures along with a binder solution using a rapid mixer granulator to obtain granules for each layer of the core;
c. drying the separate granules to obtain dried granules for each layer of the core;
d. mixing the separate dried granules with at least one excipient to obtain a final blend for each layer of the core;
e. loading the separate final blends for each layer of the core on a tableting machine;
f. compressing the final blends to form the multi-layer core of the fixed dose combination; and
g. coating the core in a coat until the weight of the coat is 1-5% of the weight of the fixed dose combination.

38. A method of preparing a fixed dose combination of Drotaverine or a pharmaceutically acceptable salt thereof and Chlordiazepoxide or a pharmaceutically acceptable salt thereof comprising a multi-layer core with a coating using a fluidized bed process, the method comprising:
a. mixing intragranular ingredients for each layer of the core separately;
b. forming granules from the mixed intragranular ingredients and a binder solution by fluidized bed granulation for each layer of the core;
c. drying the separate granules to obtain dried granules for each layer of the core;
d. mixing the separate dried granules with at least one excipient to obtain a final blend for each layer of the core;
e. loading the separate final blends for each layer of the core on a tableting machine;
f. compressing the final blends to form the multi-layer core of the fixed dose combination; and
g. coating the core in a coat until the weight of the coat is 1-5% of the weight of the fixed dose combination.

39. A method of treating at least one symptom of gastrointestinal, disorder characterized by spastic conditions of smooth muscles in a subject in need thereof comprising administering to the subject the formulation of any of claims 1-30.

40. The method of claim 39, wherein the disorder comprises irritable bowel syndrome, gastrointestinal manifestations of anxiety and tension in the gastrointestinal tract, abdominal pain, or gastrointestinal spasms secondary to organic diseases of the digestive system, or a combination thereof.

41. A pharmaceutical composition for use in treating at least one symptom of gastrointestinal disorder characterized by spastic conditions of smooth muscles in a subject in need thereof comprising administering to the subject the formulation of any of claims 1-30.

42. The pharmaceutical composition for use of claim 41, wherein the disorder comprises irritable bowel syndrome, gastrointestinal manifestations of anxiety and tension in the gastrointestinal tract, abdominal pain, or gastrointestinal spasms secondary to organic diseases of the digestive system, or a combination thereof.

43. A combination comprising Drotaverine or a pharmaceutically acceptable salt thereof and at least one benzodiazepine formulated as a single medicine.
